(19) United States
(12) Patent Application Publication
(10) Pub. No.: US 2008/0196520 A1
(43) Pub. Date: Aug. 21, 2008

(54) Methods of Determining Dispersant-Containing Contamination of Pigment and Mineral Products

(75) Inventors: Stacey Johnson, Cochran, GA (US); Edward J. Sare, Macon, GA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: IMERYS KAOLIN, INC., DRY BRANCH, GA (US)

(21) Appl. No.: 11/912,014

(22) PCT Filed: Apr. 19, 2006
(86) PCT No.: PCT/US2006/014736

§ 371 Date: Oct. 19, 2007

Related U.S. Application Data
(60) Provisional application No. 60/672,993, filed on Apr. 20, 2005.

(51) Int. Cl. G01N 33/00

(52) U.S. Cl. 73/866

(57) ABSTRACT

Disclosed herein are methods of determining the presence of contaminating dispersants, such as polymeric dispersants and inorganic dispersants, in a processed mineral, including mineral products such as pigments, by measuring the particle charge of the processed mineral using a particle charge detector.

This application claims priority to U.S. Provisional Patent Application No. 60/672,993, filed Apr. 20, 2005.

Disclosed herein are methods of determining the presence of a contaminating dispersant in a processed mineral, including pigments or other mineral products, by measuring the charge of the processed mineral.

It is known that some equipment used in processing facilities, such as baggers, screens, and the like, are usually used in the batch-wise production of a number of different products. As a result, cross-contamination may sometimes occur, wherein one product picks up residual amounts of the previously processed material having different physical or chemical characteristics, such as when a non-dispersant containing mineral is contaminated with trace amounts of a previously processed dispersant containing mineral. This cross-contamination may be detrimental to some applications, such as catalyst substrate production in, for example, the automotive substrate field, which are sensitive to the presence of even trace amounts of dispersants. The presence of the dispersants may have significant, adverse effects on the rheological characteristics of the system, for example, ceramic bodies, in which the desired level of dispersants is minimal during processing. To determine the presence of cross-contamination, certain measurements may be used, for example, measurement of particle size distribution or brightness of the mineral material. However, those methods may not have sufficient sensitivity for detecting trace amounts of cross-contamination.

Therefore, there is still a need for a method with sufficiently high degree of sensitivity for detecting trace amount of cross-contamination of particulate mineral materials, for example, in cases involving contaminating dispersants. The present inventors have surprisingly discovered that trace amount of contaminating dispersants, such as found in dispersant containing kaolin, in a pigment or mineral product can be detected by measuring the overall charge of a pigment or a particulate mineral via, for example, a particle charge detector.

As used herein, the particle charge detector includes, for example, a streaming charge detector. It is known that such a particle charge detector can be used in various applications in determining the dosage of flocculants or fixing agents, which may influence the particle charge. Streaming charge detectors have been used in applications in which the optimization of flocculants dosing is important. Non-limiting examples of such applications include in the clarification of beverages, for example, flocculation using activated silica/gelatine-flocculation, dewatering and thickening of suspensions or effluent sludges by known dosage of flocculants, separating emulsions and optimizing flocculants in sewage treatment industries, and elimination of “anionic trash” from papermill whitewater circuits in paper industry.

Particle charge detectors can also be used to control charge characteristics of polymers and other additives to optimize their efficiency and to determine stability of pigments and paints and increase their shelf lives. However, such detectors have not been used to detect contamination of dispersants in processed mineral product, including pigments and other mineral products, in systems that benefit from minimal levels of dispersant, such as in ceramic bodies used in catalyst substrate production.

Therefore, disclosed herein is a method of determining the presence of a contaminating dispersant in pigment or mineral products, comprising measuring the overall charge of a particulate mineral using a particle charge detector. The method disclosed herein can be used in the processing and/or for the final product.

The contaminating dispersant may comprise any dispersant known in the art for the dispersion of particulate minerals in an aqueous medium. In one embodiment, the contaminating dispersant comprises at least one anionic organic dispersant chosen from anionic organic polyelectrolytes. Exemplary polyelectrolytes include those comprising a polycarboxylate.

Typical polycarboxylate can be chosen from homopolymers and copolymers comprising at least one monomer residue (the portion of the polymer derived from the monomer) chosen from vinyl and olefinic groups substituted with at least one carboxylic acid group, and water soluble salts thereof. The at least one monomer residue can be derived from monomers chosen from acrylic acid, methacrylic acid, itaconic acid, chronic acid, fumaric acid, maleic acid, maleic anhydride, isocrotonic acid, undecylenic acid, angelic acid, and hydroxyacrylic acid.

In one embodiment, the polycarboxylate can have a number average molecular weight of no greater than about 20,000, as measured by the method of gel permeation chromatography using a low angle laser light scattering detector. In another embodiment, the polycarboxylate has a number average molecular weight ranging from about 700 to about 10,000.

For example, the at least one anionic dispersant is chosen from polyacrylates, such as partially and fully neutralized sodium polyacrylates. Further, for example, the at least one anionic dispersant is chosen from partially and fully neutralized maleic anhydride copolymers.

In another embodiment, the contaminating dispersant comprises at least one inorganic dispersant chosen from those commonly used in the art. For example, the at least one inorganic dispersant may be chosen from silicates such as sodium silicate, lithium silicate, and ammonium silicate. The at least one inorganic dispersant may also be chosen from water soluble condensed phosphates such as sodium hexametaphosphate, trisodium phosphate, tetrasodium phosphate, tetrasodium pyrophosphate, sodium tripolyphosphate, and sodium acid pyrophosphate.

As used herein, the processed mineral may comprise a pigment product, which includes both inorganic pigment products and organic pigment products in various forms, such as a dispersion in an aqueous medium or dry powder. Non-limiting examples of such inorganic pigments include satin white, titania, and calcium sulphate.

More generally, the processed mineral as disclosed herein may comprise a particulate inorganic material known in the art, including, for example, kaolin, such as hydrous kaolin and calcined kaolin, calcium carbonate, such as ground calcium carbonate (GCC) and precipitated calcium carbonate (PCC), talc, perlite, diatomite, dolomite, nepheline syenite, mica, and feldspar. In one embodiment, kaolin is used. PCC is generally prepared by a process in which calcium carbonate is calcined to produce calcium oxide, or “quicklime,” the quicklime then is “slaked” with water to produce an aqueous slurry of calcium hydroxide, and finally, the calcium hydroxide is carbonated with a carbon-dioxide-containing gas to produce PCC. GCC may comprise ground naturally occurring calcium carbonate from sources such as marble, limestone, and chalk. PCC may also be ground.

In addition, the mineral product as disclosed herein may comprise at least one mineral chosen from TiO₂, silica, and silicon carbide.

The mineral product as disclosed herein can also be in various forms, such as a dispersion in an aqueous medium or dry powder.

The overall charge of the foregoing processed minerals, such as the pigment or particulate minerals, is measured using a particle charge detector, such as a streaming charge detector. For example, the particle charge detector measures the zeta potential of a mineral dispersion. Such a measurement can be accomplished readily by one of ordinary skill in the art.

The present disclosure is further illuminated by the following non-limiting examples, which are intended to be purely exemplary of the disclosure. The percentages expressed below are by weight.

### EXAMPLES

In the following examples, five samples of kaolin blends (A, B, C, D, and E) were used, wherein Sample A does not have any contaminating dispersant; while Samples B, C, D, and E were kaolins that had been pre-dispersed with high levels of sodium polyacrylate dispersant. The kaolin blends in Samples B, C, D, and E are different. The physical properties of the five kaolins blend samples, including relative dispersant level, brightness, and particle size distribution, were measured.

The relative dispersant level on a dry basis was reflected by the measurement of cationic demand of the sample using Mütek® PCD 02, manufactured by Mutek Analytic, Inc. The instructions and procedures of the measurement in the User's Manual of Mütek® PCD 02 were followed. Titration was conducted manually, taking into account of the actually measured difference between the automatic titrator's initial potential (mv) reading and the isoelectric point.

The ISO brightness of the product produced by the method disclosed herein can be measured by standard methods known to one of ordinary skill in the art using, for example, a Technibrite TB1C brightness analyzer.

The particle size distribution was determined by measuring the sedimentation of the particulate sample in a fully dispersed condition in a standard aqueous medium, such as water, using a SEDIGRAPH™ instrument, e.g., SEDIGRAPH 5100, obtained from Micromeritics Corporation, USA. The “particle size” of a given particle is expressed in terms of the diameter of a sphere of equivalent diameter, which sediments through the medium, i.e., an equivalent spherical diameter (ESD). The weight percentages of the kaolin samples with an ESD of less than 10 μm, less than 5 μm, less than 2 μm, less than 1 μm, and less then 0.5 μm were measured respectively.

The physical properties of the five kaolin blend samples were summarized in Table I below.

TABLE I

| | Mütek ® | | % < | % < | % < | % < | % < |
|---|---|---|---|---|---|---|---|
| Kaolin | Measurement | ISO | 10 | 5 | 2 | 1 | 0.5 |
| Sample | (meg/g) | Brightness | μm | μm | μm | μm | μm |
| A | −39 | 84.90 | 99.3 | 96.3 | 83.9 | 67.6 | 45.4 |
| B | −150 | 90.29 | 99.8 | 99.6 | 99.1 | 98.5 | 92.9 |
| C | −175 | 87.63 | 99.3 | 96.6 | 80.5 | 65.2 | 44.9 |
| D | −195 | 88.79 | 99.5 | 98.4 | 92.1 | 81.7 | 63.5 |
| E | −220 | 85.01 | 99.2 | 95.6 | 83.6 | 72.9 | 59.1 |

### Example 1

In this example, Sample B was blended with Sample A at 10% intervals from 0 to 100%, while keeping the total weight of the mixture unchanged. The physical properties of the resulting blend, including the relative dispersant level, brightness, and particle size distribution, were measured as discussed above. The results are shown in Table II below.

TABLE II

| | | Mütek ® | | | | | | |
|---|---|---|---|---|---|---|---|---|
| % | % | Measurement | ISO | % < | % < | % < | % < | % < |
| Sample A | Sample B | (meg/g) | Brightness | 10 μm | 5 μm | 2 μm | 1 μm | 0.5 μm |
| 100 | 0 | −39 | 84.90 | 99.3 | 96.3 | 83.9 | 67.6 | 45.4 |
| 90 | 10 | −50 | 85.67 | 99.8 | 97.3 | 85.5 | 70.9 | 52.4 |
| 80 | 20 | −61 | 86.31 | 98.8 | 97.0 | 86.9 | 73.6 | 56.2 |
| 70 | 30 | −66 | 86.86 | 99.2 | 97.0 | 88.5 | 77.2 | 61.8 |
| 60 | 40 | −78 | 87.50 | 98.8 | 96.8 | 89.8 | 79.8 | 66.0 |
| 50 | 50 | −93 | 88.17 | 98.7 | 96.6 | 90.5 | 81.8 | 70.1 |
| 40 | 60 | −100 | 88.60 | 98.2 | 97.4 | 92.6 | 86.0 | 75.0 |
| 30 | 70 | −112 | 88.94 | 99.4 | 98.2 | 94.7 | 89.6 | 80.7 |
| 20 | 80 | −118 | 89.25 | 99.2 | 98.4 | 96.2 | 92.2 | 84.1 |
| 10 | 90 | −129 | 89.63 | 100.6 | 99.9 | 98.0 | 96.0 | 89.7 |
| 0 | 100 | −150 | 90.29 | 99.8 | 99.6 | 99.1 | 98.5 | 92.9 |

As shown in Table II, with the increasing amount of the contaminating dispersant, the absolute overall charge of the kaolin sample as reflected by the Mütek® measurement, the brightness, and the particle size distribution all change. Specifically, the resulting kaolin blend sample with the presence of the contaminating dispersant has more overall charge, higher brightness, and finer particle size than Sample A, which does not have the contaminating dispersant. However, the increase of the absolute overall charge of the resulting kaolin blend sample is more obvious than the changes in the brightness and the particle size distribution.

### Example 2

In this example, Sample C was blended with Sample A at 20% intervals from 0 to 100%, while keeping the total weight of the mixture unchanged. The physical properties of the resulting blend, including the relative dispersant level, brightness, and particle size distribution, were measured as discussed above. The results are shown in Table III below.

TABLE III

| | | Mütek ® | | | | | | |
|---|---|---|---|---|---|---|---|---|
| % | % | Measurement | ISO | % < | % < | % < | % < | % < |
| Sample A | Sample C | (meg/g) | Brightness | 10 μm | 5 μm | 2 μm | 1 μm | 0.5 μm |
| 100 | 0 | −39 | 84.90 | 99.3 | 96.3 | 83.9 | 67.6 | 45.4 |
| 80 | 20 | −60 | 85.64 | 99.2 | 96.2 | 82.7 | 66.8 | 46.6 |
| 60 | 40 | −85 | 86.19 | 99.8 | 96.8 | 82.7 | 66.8 | 47.3 |
| 40 | 60 | −100 | 86.78 | 99.5 | 96.6 | 82.3 | 66.6 | 46.7 |
| 20 | 80 | −125 | 87.17 | 100.1 | 97.5 | 81.7 | 65.7 | 46.9 |
| 0 | 100 | −175 | 87.63 | 99.3 | 96.6 | 80.5 | 65.2 | 44.9 |

As shown in Table III, with the increasing amount of the contaminating dispersant, the absolute overall charge of the resulting kaolin blend sample as reflected by the Mütek® measurement and the brightness change; while the particle size distribution remains similar. Specifically, the resulting kaolin blend sample with the presence of the contaminating dispersant has more overall charge and slightly higher brightness than Sample A, which does not have the contaminating dispersant. However, the increase of the absolute overall charge of the resulting kaolin blend sample is more obvious than the change in the brightness.

### Example 3

In this example, Sample D was blended with Sample A at 10% intervals from 0 to 100%, while keeping the total weight of the mixture unchanged. The physical properties of the resulting blend, including the relative dispersant level, brightness, and particle size distribution, were measured as discussed above. The results are shown in Table IV below.

TABLE IV

| | | Mütek ® | | | | | | |
|---|---|---|---|---|---|---|---|---|
| % | % | Measurement | ISO | % < | % < | % < | % < | % < |
| Sample A | Sample D | (meg/g) | Brightness | 10 μm | 5 μm | 2 μm | 1 μm | 0.5 μm |
| 100 | 0 | −39 | 84.90 | 99.3 | 96.3 | 83.9 | 67.6 | 45.4 |
| 90 | 10 | −45 | 85.48 | 99.1 | 97 | 84.9 | 69.3 | 49.1 |
| 80 | 20 | −64 | 85.63 | 98.8 | 96.9 | 85.2 | 70.5 | 51.6 |
| 70 | 30 | −70 | 86.18 | 99.3 | 96.7 | 86.4 | 71.5 | 52.3 |
| 60 | 40 | −80 | 86.44 | 99.5 | 97.8 | 87.7 | 73.6 | 54.0 |
| 50 | 50 | −100 | 87 | 98.8 | 97 | 87.4 | 74.5 | 55.5 |
| 40 | 60 | −118 | 87.27 | 99.7 | 98 | 89.3 | 76.0 | 57.7 |
| 30 | 70 | −131 | 87.7 | 100.1 | 98.7 | 90.5 | 78.1 | 59.7 |
| 20 | 80 | −155 | 88.11 | 99.3 | 98.4 | 91.5 | 79 | 60.7 |
| 10 | 90 | −170 | 88.52 | 100.5 | 99.4 | 92.7 | 81.0 | 62.8 |
| 0 | 100 | −195 | 88.79 | 99.5 | 98.4 | 92.1 | 81.7 | 63.5 |

As shown in Table IV, with the increasing amount of the contaminating dispersant, the absolute overall charge of the resulting kaolin blend sample as reflected by the Mütek® measurement, the brightness, and the particle size distribution all change. Specifically, the resulting kaolin blend sample with the presence of the contaminating dispersant has more overall charge, higher brightness, and finer particle size than Sample A, which does not have the contaminating dispersant. However, the increase of the absolute overall charge of the resulting kaolin blend sample is more obvious than the changes in the brightness and the particle size distribution.

### Example 4

In this example, Sample E was blended with Sample A at 20% intervals from 0 to 100%, while keeping the total weight of the mixture unchanged. The physical properties of the resulting blend, including the relative dispersant level, brightness, and particle size distribution, were measured as discussed above. The results are shown in Table V below.

TABLE V

| | | Mütek ® | | | | | | |
|---|---|---|---|---|---|---|---|---|
| % | % | Measurement | ISO | % < | % < | % < | % < | % < |
| Sample A | Sample E | (meg/g) | Brightness | 10 μm | 5 μm | 2 μm | 1 μm | 0.5 μm |
| 100 | 0 | −39 | 84.90 | 99.3 | 96.3 | 83.9 | 67.6 | 45.4 |
| 80 | 20 | −65 | 85.08 | 98.3 | 95.6 | 82.5 | 67 | 47.3 |
| 60 | 40 | −97 | 85.02 | 99.2 | 96.6 | 82.6 | 69.4 | 51.3 |
| 40 | 60 | −125 | 84.95 | 99.3 | 96.4 | 83.5 | 70.8 | 54.5 |
| 20 | 80 | −158 | 85.01 | 98.4 | 95.4 | 82.9 | 71.2 | 55.8 |
| 0 | 100 | −220 | 85.01 | 99.2 | 95.6 | 83.6 | 72.9 | 59.1 |

As shown in Table V, with the increasing amount of the contaminating dispersant, the absolute overall charge of the resulting kaolin blend sample as reflected by the Mütek® measurement and the small particle size distribution (i.e., weight percentages of kaolin particles with an ESD of less than 1 μm and less than 0.5 μm) change; while the brightness and the large particle size distribution remain similar. Specifically, the resulting kaolin blend sample with the presence of the contaminating dispersant has more overall charge and slightly finer particles than Sample A, which does not have the contaminating dispersant. However, the increase of the absolute overall charge of the resulting kaolin blend sample is more obvious than the change in the particle size distribution.

As shown in Tables II-V, even for different types of kaolins, i.e., kaolin blend samples B-E, the increase of the absolute overall charge of the resulting kaolin blend sample is more obvious than the change in other physical properties, such as the brightness and the particle size distribution.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Other embodiments of the invention will be apparent to those skilled in the art from consideration of the specification. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of determining the presence of a contaminating dispersant in a processed mineral comprising measuring the particle charge of the processed mineral using a particle charge detector.

2. The method according to claim 1, wherein the contaminating dispersant is chosen from polymeric dispersants.

3. The method according to claim 2, wherein the polymeric dispersants comprise at least one anionic organic dispersant chosen from anionic organic polyelectrolytes.

4. The method according to claim 3, wherein the anionic organic polyelectrolytes comprise at least one polycarboxylate chosen from homopolymers and copolymers comprising at least one monomer residue chosen from vinyl and olefinic groups substituted with at least one carboxylic acid group, and water soluble salts thereof.

5. The method according to claim 4, wherein the at least one monomer residue can be derived from monomers chosen from acrylic acid, methacrylic acid, itaconic acid, chronic acid, fumaric acid, maleic acid, maleic anhydride, isocrotonic acid, undecylenic acid, angelic acid, and hydroxyacrylic acid.

6. The method according to claim 2, wherein the polymeric dispersants are chosen from polyacrylates.

7. The method according to claim 1, wherein the contaminating dispersant is chosen from inorganic dispersants.

8. The method according to claim 7, wherein the inorganic dispersants are chosen from silicates and water soluble condensed phosphates.

9. The method according to claim 8, wherein the silicates are chosen from sodium silicate, lithium silicate, and ammonium silicate.

10. The method according to claim 8, wherein the water soluble condensed phosphates are chosen from sodium hexametaphosphate, trisodium phosphate, tetrasodium phosphate, tetrasodium pyrophosphate, sodium tripolyphosphate, and sodium acid pyrophosphate.

11. The method according to claim 1, wherein the processed mineral comprises at least one pigment or mineral product.

12. The method according to claim 11, wherein the at least one pigment or mineral product is in a form of a dispersion in an aqueous medium or dry powder.

13. The method according to claim 11, wherein the pigment is chosen from inorganic pigments and organic pigments.

14. The method according to claim 11, wherein the mineral product comprises a kaolin.

15. The method according to claim 11, wherein the mineral product comprises at least one mineral chosen from calcium carbonate and dolomite.

16. The method according to claim 11, wherein the mineral product comprises at least one mineral chosen from talc, perlite, diatomite, nepheline syenite, mica, feldspar, TiO₂, silica, and silicon carbide.

17. The method according to claim 1, wherein the particle charge detector is a streaming charge detector.

18. The method according to claim 1, wherein the particle charge detector measures the zeta potential of a mineral dispersion.
